(19) United States
(12) Patent Application Publication
(10) Pub. No.: US 2020/0101029 A1
(43) Pub. Date: Apr. 2, 2020

(54) METHOD AND SYSTEM FOR PRESERVING COGNITIVE CAPACITY DURING AGING

(71) Applicant: James E. Beecham, West Palm Beach, FL (US)

(72) Inventor: James E. Beecham, West Palm Beach, FL (US)

(21) Appl. No.: 16/210,890

(22) Filed: Dec. 5, 2018

Related U.S. Application Data
(60) Provisional application No. 62/772,106, filed on Nov. 28, 2018.
(60) Provisional application No. 62/771,113, filed on Nov. 25, 2018.
(60) Provisional application No. 62/771,037, filed on Nov. 24, 2018.
(60) Provisional application No. 62/771,034, filed on Nov. 24, 2018.
(60) Provisional application No. 62/770,874, filed on Nov. 23, 2018.
(60) Provisional application No. 62/770,808, filed on Nov. 22, 2018.
(60) Provisional application No. 62/770,649, filed on Nov. 21, 2018.
(60) Provisional application No. 62/770,259, filed on Nov. 21, 2018.
(60) Provisional application No. 62/770,193, filed on Nov. 21, 2018.
(60) Provisional application No. 62/770,190, filed on Nov. 21, 2018.
(60) Provisional application No. 62/770,116, filed on Nov. 20, 2018.
(60) Provisional application No. 62/769,706, filed on Nov. 20, 2018.
(60) Provisional application No. 62/769,353, filed on Nov. 19, 2018.
(60) Provisional application No. 62/769,003, filed on Nov. 19, 2018.
(60) Provisional application No. 62/768,925, filed on Nov. 18, 2018.
(60) Provisional application No. 62/768,880, filed on Nov. 17, 2018.
(60) Provisional application No. 62/768,122, filed on Nov. 16, 2018.
(60) Provisional application No. 62/767,084, filed on Nov. 14, 2018.
(60) Provisional application No. 62/760,661, filed on Nov. 13, 2018.
(60) Provisional application No. 62/758,662, filed on Nov. 11, 2018.
(60) Provisional application No. 62/748,484, filed on Oct. 21, 2018.
(60) Provisional application No. 62/746,655, filed on Oct. 17, 2018.
(60) Provisional application No. 62/742,293, filed on Oct. 6, 2018.
(60) Provisional application No. 62/739,942, filed on Oct. 2, 2018.

(51) Int. Cl. A61K 31/132; A61K 45/06; A61P 25/28; A61K 35/745; A61K 31/26; A61K 31/353; A61K 36/185; A23L 33/105; A23L 33/135; A23C 19/072

(52) U.S. Cl. CPC A61K 31/132; A61K 45/06; A61P 25/28; A61K 35/745; A61K 31/26; A61K 2035/115; A61K 36/185; A23L 33/105; A23L 33/135; A23C 19/072; A61K 31/353

(57) ABSTRACT

The present invention comprises compositions having a.) at least one acetyltransferase EP300 inhibitor-substance, such as chemically pure spermidine or spermidine-rich wheat germ extract, and optionally b.) at least one nrf2 activator-substance, such as cocoa flavonoids. In use, a user typically adds said compositions to food during the day, and then consumes compositions with food. Typically, such additions are scheduled to occur regularly on consecutive days, for months and over years, such as edible bits of sprinkles-configuration added to meals and snacks, or other configurations as herein disclosed. Such compositions are typically consumed by user in divided doses each day, as disclosed herein.

### CROSS-REFERENCE TO RELATED APPLICATIONS

10011 The present application claims priority to U.S. Provisional Application Ser. No. 62/772,106 filed on Nov. 28, 2018 entitled “METHOD AND SYSTEM FOR PRESERVING COGNITIVE CAPACITY DURING AGING”, U.S. Provisional Application Ser. No. 62/771,113 filed on Nov. 25, 2018 entitled “METHOD AND SYSTEM FOR PRESERVING COGNITIVE CAPACITY DURING AGING”, U.S. Provisional Application Ser. No. 62/771,037 filed on Nov. 24, 2018 entitled “METHOD AND SYSTEM FOR PRESERVING COGNITIVE CAPACITY DURING AGING”, U.S. Provisional Application Ser. No. 62/771,034 filed on Nov. 24, 2018 entitled “METHOD AND SYSTEM FOR PRESERVING COGNITIVE CAPACITY DURING AGING”, U.S. Provisional Application Ser. No. 62/770,874 filed on Nov. 23, 2018 entitled “METHOD AND SYSTEM FOR PRESERVING COGNITIVE CAPACITY DURING AGING”, U.S. Provisional Application Ser. No. 62/770,808 filed on Nov. 22, 2018 entitled “METHOD AND SYSTEM FOR PRESERVING COGNITIVE CAPACITY DURING AGING”, U.S. Provisional Application Ser. No. 62/770,649 filed on Nov. 21, 2018 entitled “METHOD AND SYSTEM FOR PRESERVING COGNITIVE CAPACITY DURING AGING”, U.S. Provisional Application Ser. No. 62/770,259 filed on Nov. 21, 2018 entitled “METHOD AND SYSTEM FOR PRESERVING COGNITIVE CAPACITY DURING AGING”, U.S. Provisional Application Ser. No. 62/770,193 filed on Nov. 21, 2018 entitled “METHOD AND SYSTEM FOR PRESERVING COGNITIVE CAPACITY DURING AGING”, U.S. Provisional Application Ser. No. 62/770,190 filed on Nov. 21, 2018 entitled “METHOD AND SYSTEM FOR PRESERVING COGNITIVE CAPACITY DURING AGING”, U.S. Provisional Application Ser. No. 62/770,116 filed on Nov. 20, 2018 entitled “METHOD AND SYSTEM FOR PRESERVING COGNITIVE CAPACITY DURING AGING”, U.S. Provisional Application Ser. No. 62/769,706 filed on Nov. 20, 2018 entitled “METHOD AND SYSTEM FOR PRESERVING COGNITIVE CAPACITY DURING AGING”, U.S. Provisional Application Ser. No. 62/769,353 filed on Nov. 19, 2018 entitled “METHOD AND SYSTEM FOR PRESERVING COGNITIVE CAPACITY DURING AGING”, U.S. Provisional Application Ser. No. 62/769,003 filed on Nov. 19, 2018 entitled “METHOD AND SYSTEM FOR PRESERVING COGNITIVE CAPACITY DURING AGING”, U.S. Provisional Application Ser. No. 62/768,925 filed on Nov. 18, 2018 entitled “METHOD AND SYSTEM FOR PRESERVING MEMORY CAPACITY DURING AGING”, U.S. Provisional Application Ser. No. 62/768,880 filed on Nov. 17, 2018 entitled “METHOD AND SYSTEM FOR PRESERVING MEMORY CAPACITY DURING AGING”, U.S. Provisional Application Ser. No. 62/768,122 filed on Nov. 16, 2018 entitled “METHOD AND SYSTEM FOR PRESERVING MEMORY CAPACITY DURING AGING”, U.S. Provisional Application Ser. No. 62/767,084 filed on Nov. 14, 2018 entitled “METHOD AND SYSTEM FOR PRESERVING MEMORY CAPACITY DURING AGING”, U.S. Provisional Application Ser. No. 62/760,661 filed on Nov. 13, 2018 entitled “METHOD AND SYSTEM FOR PRESERVING MEMORY CAPACITY DURING AGING”, U.S. Provisional Application Ser. No. 62/758,662 filed on Nov. 11, 2018 entitled “METHOD AND SYSTEM FOR PRESERVING MEMORY CAPACITY DURING AGING”, U.S. Provisional Application Ser. No. 62/748,484 filed on Oct. 21, 2018 entitled “Method and System for Memory preservation in poor diet/habit population”, U.S. Provisional Application Ser. No. 62/746,655 filed on Oct. 17, 2018 entitled “Method and System to preserve brain gray matter”, U.S. Provisional Application Ser. No. 62/742,293 filed on Oct. 4, 2018 entitled “Method and System to improve compliance to Anti-Alzheimer's disease (AAD) diet”, U.S. Provisional Application Ser. No. 62/739,942 filed on Oct. 2, 2018 entitled “Engendering hope in Alzheimer's disease patients”, the disclosures of which are hereby incorporated in their entirety at least by reference.

### BACKGROUND OF THE INVENTION

### 1. Field of the Invention

The present invention generally relates to methods and systems for preventing deterioration during aging of human cognitive function, more particularly memory function. Aging is the factor most closely associated to reduction in memory capacity in humans, more particularly in working memory loss and declarative memory loss. Declarative memory is to recall fact and events. Working memory is to carry out normal daily cognitive tasks like language comprehension, reading, learning, or reasoning. It is the deterioration of such normal memory functions which is a prominent feature of common dementias such as Alzheimer's disease.

### 2. Description of Related Art

Current scientific understanding of Alzheimer's disease focuses on linking the deterioration of brain function to brain pathology, such as to which neurons have died in the brain, and to which abnormal proteins have accumulated in the brain. Among these abnormal proteins known to accumulate in Alzheimer's disease within the human brain are amyloid beta protein and phosphorylated tau protein.

Current scientific understanding of pharmaceutical treatment for Alzheimer's disease tends to focus on neurotransmitter acetylcholine levels, and more particularly on medicines directed to blocking enzyme acetylcholinesterase, an enzyme which degrades acetylcholine. In research on animals pertaining to Alzheimer's disease, the focus tends to be to attempt interventions by which researchers hope to prevent, reverse or at least reduce the amount of abnormal protein accumulations in the brain.

Nevertheless, it is well known that Alzheimer's disease continues to be an expanding major health problem. Projections are that the number of persons affected will increase by millions of persons over the coming decades. Treatments currently approved for use in Alzheimer's disease are generally far less than satisfactory.

### BRIEF SUMMARY OF THE INVENTION

In one embodiment, the present invention comprises compositions having a.) at least one acetyltransferase EP300 inhibitor substance such as chemically pure spermidine or spermidine-rich wheat germ extract, and b.) optionally composition also comprises at least one nrf2 activator substance such as cocoa flavonoids. In use, a user typically adds said compositions to food during the day, and then consumes compositions with food. Typically, such additions are scheduled to occur regularly on consecutive days, for months and over years, as edible bits of sprinkles-configuration added to meals and snacks, or other configurations as herein disclosed. Such compositions are typically consumed by user in divided doses each day, as disclosed herein. Dose amounts typically approximate a calculated peak physiology amount. This is because research has indicated that a dose-response curve of sorts operates inside brain cells (Brain Res. 1993 Mar. 12; 605(2):322-6) regarding spermidine concentrations, whereby beneficial pathways are activated by lower/physiologic concentrations of spermidine (European Journal of Pharmacology 684 (2012) 79-86) but those same pathways might be inhibited by higher concentration of spermidine.

It is a goal of the present invention to bring levels of spermidine via supplementation to be within the range in neurons and blood concentration as seen over aging years in those living to be centenarians or supercentenarians or the like, as such concentration can logically be judged by reference to longevity studies, as herein disclosed. Excess or extra-physiologic concentration of spermidine is not a goal herein.

Studies of substances capable to inhibit acetyltransferase EP300 have revealed their biologic ability to boost partial autophagy within cells, i.e. the process that degrades and removes abnormal or excess protein from within cells. Studies of nrf2 activator substances have revealed their biological ability to induce increased production of natural anti-oxidants within cells, such as boosting glutathione levels.

The adding to user foods of spermidine in doses disclosed, and optionally the adding simultaneously of the nrf2 activator substance to food, and then user consuming such with food, as disclosed within instant invention, has the capacity to decrease levels of damaging abnormal protein within same cells while optionally simultaneously raising natural anti-oxidant levels within said cells. For brain cells in particular, such actions are beneficial, tending to preserve cognitive capacity, especially if carried out daily for years in doses matched to timing of/amount of food consumption, as herein disclosed to be done. This pattern of consumption of herein disclosed of formulations/compositions holds potential for a user to preserve memory and preserve cognitive ability during years of aging.

Scientific research has discovered at least one reason why removing such abnormal protein (Redox Biology 14 (2018), 450-464) from within brain cells is protective for cognition. Studies have shown that such abnormal protein, if not removed daily, can bind to metal ions within the cell cytoplasm, and become misfolded. Such misfolded protein/metal ion combinations have been shown to cause production of damaging reactive oxygen species within cells. Where natural anti-oxidant levels are low within neurons, especially those neurons simultaneously facing an increase in reactive oxygen species from misfolded protein/metal ion combinations, the damage to memory capacity slowly and relentlessly goes forward, day after day.

A high rate of oxidative damage within neurons, and a low level of anti-oxidant protection inside neurons is precisely the situation which tends to occur daily within brain cells of a person consuming the unhealthy food and excess calories of the standard fast food diet. In one embodiment of the instant invention, the instant inventor discloses benefit in timing of user consuming the compositions herein disclosed as key. This is because instant inventor perceives the timing of the actions of the active ingredients within the compositions, at least one nrf2 activator and at least one acetyltransferase EP300 inhibitor, to timely boost natural pathways to counteract the potential for damage which occurs during the mealtime user consumption of low-nutrient/high calorie food/snacks/meals. It appears to the instant inventor that it is the high-calorie, low-nutrient food consumptions of typical fast food diet which tends to exacerbate the aging-related deficit to cognitive function and memory derived in aging. Such deficits are intensified by fast food having low levels of the nrf2 activator substances, and having low levels in acetyltransferase EP300 inhibitors. This ‘anti-oxidant deficit/increase in abnormal protein’ circumstance allows reactive oxygen species to overcome the defense of anti-oxidants and instead damage the neuron from the inside, day after day, contributing to decline in cognitive capacity over the years. The instant invention timings and active ingredients and amounts are disclosed to counteract such aging-related deficits from occurring.

In one embodiment, the instant invention at least partially corrects this double defect situation, by providing nrf2 activators and acetyltransferase EP300 inhibitors in at least approximately the ‘gap’ or deficit amounts in a particular user's diet. Furthermore, the instant invention provides such supplemented amounts in divided doses over the day, so as to match the times of food intake. The benefit of such matched-to-user amounts and timings is that the protection needed is disclosed herein to be provided at the most acute moments, i.e. during time of high metabolic activity. This approach is suggested by studies demonstrating a longevity boost in those individuals on calorie restriction diets. When individual avoids high metabolic times, the cell is protected. With modern diets, such high metabolic times are unavoidable, thus the instant invention is offered. In one embodiment, the dosage times of consumables herein disclosed matches the times when most needed by brain cells for maximum resistance to damage. In one embodiment, the instant invention further provides benefit to user via providing user access to a grounding device, a means for user to lower his blood viscosity, to the further benefit of delivery of said nrf2 activators and acetyltransferase EP300 inhibitors to user's brain cells.

In one embodiment of the instant invention, a user whose diet is deficient in nrf2 activators and deficient in acetyltransferase EP300 inhibitors will herein derive benefit via improved intake of each acetyltransferase EP300 inhibitor and nrf2 activator simultaneously. In one embodiment of the instant invention, such a beneficially supplied user will contemporaneously improve blood flow to his brain via thinning of blood viscosity as accomplished by use of a grounding device, as herein disclosed. In one embodiment, such a user thus has a synergistic improvement to cognitive protection, as a result of simultaneously increased supply of both nrf2 activator and acetyltransferase EP300 inhibitor within the herein supplemented food as consumed by user, plus the contemporaneous synergistic benefit to user's blood flow rate by lower blood viscosity, thus boosting brain delivery of such beneficial substances. Thus, more of the said supplemented activators and said supplemented inhibitors will be delivered to brain cells at brain cells' maximum time of need for protection from excess metabolism-induced damage.

In one embodiment, a variety of compositions are provided to user as to dose of active ingredient within composition, and as to timing recommended to consume, such as one pill with snack, two pills with small meals, and the like. In one embodiment, said choice is advised by expert staff/expert system. Such advice as to which compositions a user might most beneficially use, and as to the amounts of nrf2 activator and/or amounts of inhibitor of acetyltransferase EP300 selected to be most beneficial to a particular user during a day, these selections are based, in one embodiment, on defining specific gaps in user's diet. In one embodiment, such defining or identification of said gaps of dietary intake of nrf2 activators and/or dietary intake of inhibitors of acetyltransferase EP300 is accomplished via user being enabled to supply details to system of user's recent diet consumptions. Such details are then rendered by system into estimation of specific user's gaps in diet, such gaps defined as deficits in consumption by comparison of user diet specifics versus ideal diet specific consumption levels or versus published data from population studies for specific substance consumption levels on average. In one embodiment, such identification of gaps is accomplished via use of algorithm. An example of such an algorithm is in FIG. 8, as will be further discussed below. In one embodiment, the instant invention provides daily to user at least approximately the gap amount as total daily dose, as thus determined to be missing in user diet. Such total daily dose is typically divided and allocated per time and amount of calorie consumption by user. Such total daily dose is typically as to nrf2 activators consumption and/or consumption of inhibitors of acetyltransferase EP300 consumption. In one embodiment, such filling of dietary gaps for user, such as by user consuming targeted compositions as herein formulated, such filling of gaps is synergistically facilitated by rendering user's blood viscosity into low-normal blood viscosity range via concomitant use by user of grounding device.

In one embodiment, user is enabled to add gap-filling compositions as sprinkles to food, in amount of sprinkles by approximate number onto the amount of calories to be consumed, at the time of consumption. Such sprinkles are then consumed with the food by user in such divided doses over the day, and in total daily dose equal to the gap amount missing from user's diet for the said substance. Such foods to which user daily applies said sprinkles optionally comprise those fast foods known to be low in the nrf2 activators and/or inhibitors of acetyltransferase EP300. Such fast foods typically comprise smoothies/shakes/fast food edible dishes and the like. In one embodiment, a user supplements in said manner daily using herein disclosed compositions so as to fill said gaps as disclosed herein, and daily over months and daily over years. Thus user receives benefits to brain cell health over the long term, despite the unhealthy diet. During these months and years of daily consuming of gap-filling compositions, said user's brain cells have protective pathways activated, as will be further disclosed herein. In one embodiment, said user also consumes via daily grounding, the earth-negative energy over the same months and years, boosting the benefit of the user consuming the said compositions.

Biologic pathways of human cellular metabolism evolved to beneficially use the substances in the food as was then available many thousands of years ago. Thus evolution, in order to accomplish survival of the fittest in the case of humans, applied available substances within natural chemistry. Thus, such foods today having natural nrf2 activators, and such foods today having natural acetyltransferase EP300 inhibitors are still key to directing human cells into said evolved protective pathways of metabolism. Such foods are still available today, but these foods are not chosen to be eaten by a large portion of humanity. It seems the reason such beneficial foods are not consumed is because other more appealing food choices have been crafted by modern taste-engineers. Furthermore, the low cost of such tastier but high calorie/low nutrient foods makes matters worse, in the sense that such low cost further leads millions of people into deficiencies in nrf2 activators consumption daily, and deficiencies in needed acetyltransferase EP300 inhibitors consumption daily. If not corrected, such deficiencies block brain cells from using the beneficial pathways otherwise available from evolution. This deficiency in consumption produces cell damage in brain neurons which continues day after day in those not consuming the healthy food substances.

In one embodiment, such pathways are returned to active and beneficial operation within brain cells of user. Such beneficial operation, as might be described as ‘programmed’ to occur by evolution, is herein enabled daily via amount, timing and substances to fill dietary gaps. In one embodiment, the synchronous sufficient supply of nrf2 substantial-activators and/or sufficient supply of acetyltransferase EP300 substantial-inhibitors daily is part of what accomplishes this revival of beneficial pathway activation for users. But, many if not most people today are unaware of the chemistry, and in any case appear to prefer foods of high taste, low cost, but deficient nutrition. In one embodiment, this common disadvantageous tendency to consume unhealthy foods of high taste is turned to advantage by making compositions herein tasty. In one embodiment, the supply herein supplemented comprising nrf2 substantial-activators and/or acetyltransferase EP300 substantial-inhibitors is comprised with confection of high flavor and satisfying taste to a specific user preference. This is accomplished by formula comprising sweetener, taste enhancers and the like. Thus, a disadvantage is turned to an advantage when a user selects a ‘taco-flavored’ composition herein, or a ‘chocolate delight’ composition herein. As a user sprinkles such flavored composition sprinkles onto his low nutrient pizza or cookie or other low-nutrient/high calorie food, and then user consumes said sprinkles/food combination. In this manner, his brain cells will be receiving chemical signals setting brain cell metabolism towards more beneficial pathways, even as his taste buds are appeased. In one embodiment, said beneficial pathways provide more anti-oxidants inside brain cells and/or provide simultaneously for a reduction in abnormal protein within cells, which equates to a reduction in damaging reactive oxygen species within brain cells. In one embodiment, this inter-neuronal ‘increased natural anti-oxidants/decreased reactive oxygen species’ status, as herein enabled to be more closely maintained daily for years in user, promotes preservation of cognitive capacity for user.

In one embodiment of the instant invention, in addition to the consumption of edible bits disclosed herein, the system further comprises a blood thinning device used by the user. The contemporaneous daily use of a grounding device, contemporaneous to user's daily consumption of the nrf2 substantial-activators/or acetyltransferase EP300 substantial-inhibitors, allows even more beneficial biologic pathways to operate inside brain cells of user. This system of consumption of calorie-consumption-timed/amounts-to-fill-gaps compositions, along with contemporaneous consumption by user of earth-negative energy, this combination of consumptions synergistically and contemporaneously boosts user cognitive function preservation. In one embodiment, user is advised by expert staff/expert system in use contemporaneously of said blood thinning device. In one embodiment, such advice is contemporaneous to advice to user, based on algorithm-revealed-gaps in diet, to add targeted composition sprinkles of edible bits containing that specific user's needed amount of nrf2 substantial-activators and/or acetyltransferase EP300 substantial-inhibitors to food daily, in during-the-day timed to calorie consumption and calorie amounts dosages, as consumed with the food.

When blood viscosity is elevated, blood flow to the brain is decreased. This is a common pattern with aging, especially over age 60. A decrease in blood flow to the brain leads to a decrease in delivery of nrf2 substantial-activators and acetyltransferase EP300 substantial-inhibitors. Such decreased delivery makes protecting cognitive function less likely. In one embodiment, user has a choice of grounding devices to select for personal use within instant invention to maintain low-normal blood viscosity.

In one embodiment, the instant invention assesses users by survey of their diet. For example, expert staff and/or an expert computer system comprised of computer hardware/software and communications technology as well-known in the art, accepts data from user regarding user's daily food intakes, such as over a week's worth of dietary reporting. Analysis is carried out by said expert staff/expert system, and as described for example in figure herein. Where it is found that user suffers from a low daily average intake of nrf2 substantial-activators and/or a low daily average intake by user for acetyltransferase EP300 substantial-inhibitors, corrective action is herein advised as disclosed and provided to user (see FIG. 8). In one embodiment, such corrective action comprises formulation of user-specific composition advisory so as to allow user to consume most beneficially daily and thus correct such specific user dietary gaps ongoing.

In one embodiment, the system comprises a.) at least one container of sprinkles matching composition needed by user, as herein disclosed to be determined by analysis of dietary gaps in user diet, and correspondingly formulated and advised, and these bits are optionally b.) carried in a shaker, and c.) such bits are several times per day added by user to daily food by said bits being shaken out from shaker container onto food in amounts and timing advised, and d.) bits so applied to food are consumed by user along with consumption of said food daily, and e.) a blood thinning device is advised to user and used frequently by user, such as daily, and f.) said device assists improvement of cerebral blood flow in user, and g.) said improved blood flow combines via said increased intake of active ingredients in edible bits so as to synergistically boost delivery to brain cells of the beneficial ingredients nrf2 substantial-activators and substantial-inhibitors of acetyltransferase EP300, and h.) user brain cells are better protected via enabled natural pathways so as to preserve cognitive capacity during aging.

In one embodiment, user is encouraged to get medical pre-clearance for user to participate in consuming edible bits herein. In one embodiment, user is encouraged to get his medical advisor to pre-clear user to use grounding device as advised herein. Such pre-clearances are viewed as useful because of possible status in user of pre-existing medical conditions. For example, a user might be taking prescribed medication to thin user's blood. The medical advisor might advise against the grounding device use on that medication basis.

Spermidine, a strong natural inhibitor of acetyltransferase EP300, has recently been reported in results of a prospective study (American Journal of Clinical Nutrition, Volume 108, Issue 2, 1 Aug. 2018, Pages 371-380) to be linked to improved longevity. In the study it was found that those humans having the highest third of intake of spermidine-rich foods over the years were statistically linked to approximately six years longer survival, and this was after the researchers excluded as best they could, influence of all other relevant factors in the diet and lifestyle. Furthermore, in one study (Rejuvenation Res. 2012 Dec. 15(6):590-5), whole blood spermidine concentration in healthy humans of both genders aging between 31 and 106 years was analyzed. In the Group 1 (age range, 31-56 years) the spermidine median [95% CI interval] concentration was approximately 63 [48-100] pmol/mg whole blood proteins. In the Group 2 (age range, 60-80 years) the spermidine concentration was approximately 23 [20-30] pmol/mg protein, i.e., about three times lower than in Group 1. In the Group 3 (age range, 90-106 years) the spermidine concentration was approximately 70 [48-81] pmol/mg protein, i.e., even higher than in Group 1. These results suggested to the study authors that increased spermidine concentration in blood was associated with longevity. Furthermore, data suggesting that spermidine supplementation is safe and excellently tolerated have been derived in both murine models and a human cohort study (Aging (Albany N.Y.). 2018 January; 10(1): 19-33). Meanwhile, a rare medical report has suggested prostate cancer patients do slightly better on a diet which restricts polyamines, a category of substances which includes spermidine. A medical advisor thus may face the circumstance wherein his patient, a prospective user of the instant invention, may request from his medical advisor a pre-clearance while said user is under treatment for prostate cancer. Such a request to said medical advisor in said circumstance can result in medical advisor recommending against said user participating in instant invention as regards consuming of spermidine-comprised consumables (Glob J Pharmaceu Sci. 2017; 2(1): 555576).

In one embodiment, the composition of the edible bits comprises, as nrf2 activator, cocoa flavonoids. In one embodiment, the composition of the edible bits, as to inhibitors of acetyltransferase EP300, comprises pharmaceutical-grade spermidine or spermidine-rich foods and/or extracts thereof and/or pure spermidine, or a spermidine analogue, such as N¹-methylspermidine. Examples of formulations are below.

In one embodiment, the instant invention system comprises expert staff. Typically such expert staff are trained, optionally certified, in the herein disclosed system, method and theory. Expert staff efficiently assist users, as will be disclosed below. Expert staff may also use algorithms and expert computer software systems, such as to compute gaps in user diet, such as for nrf2 activators and/or acetyltransferase EP300 inhibitor consumption. In one embodiment, the larger the gap, then the greater is the amount in daily total advised to user as supplement, and the timing and amount in divided dose of compositions to be consumed daily matches the time and amount of calorie consumption during the day by user.

In one embodiment, users are advised, monitored, instructed, and reasonably assessed for compliance. In one embodiment, such assisting/facilitating applies to both the edible bits selection/supply, and to the user's access to earth negative energy from grounding device use.

In one embodiment, users benefit from instruction/information, such as regarding consumables and/or use of the various grounding devices. In one embodiment, expert staff inform users that formulations of edible bits herein can be adjusted to their taste preferences, and users so indicate and apply taste preference.

In one embodiment, user benefits from a reminder via the system of the instant invention as to various aspects, such as alert to user when the time is approaching for scheduled use of aspects of the invention. An alert may remind user to use the grounding device. An alert may remind user to take edible bits to work, so as to apply bits to noon meal or the like. In one embodiment, users benefit from instructions on how many edible bits to consume per which size meal or snack. The system optionally instructs user as to amounts needed per day to fill a daily dietary gap, and the timing of that daily consumption as divided doses per instant invention formula. Such help is typically provided by expert staff/expert computer software system.

In one embodiment, said edible bits are formulated with taste enhancers, sweetener, filler and the like as is well known in the art. Optionally, the formulation is configured as pill, tablet, capsule, pharmaceutical, sprinkle, spread, snack, food item or the like, using methods well known in the art.

In one embodiment, expert staff asks user about details of user's current diet. From such a review of user's recent intake of food and beverages, an estimate is optionally undertaken to determine which composition will best serve the user as to preservation of cognitive function.

In one embodiment, for a user reporting to expert staff a frequent consumption of unhealthy fast food, especially a user stating he or she plans to continue such pattern of eating unhealthy food, such a user receives advice as per system herein, such as opportunity to choose a stronger dose composition of active ingredients nrf2 activator/inhibitor of acetyltransferase EP300 to fill anticipated gap. In one embodiment, users ‘with a sweet tooth’, receive a suggestion from staff expert to try the sweetened sprinkle compositions.

In one embodiment, for a user who reports to expert staff that he/she is already on the Mediterranean diet, and faithfully eats such diet, staff expert may advise a lower dose daily of edible bits during that time of improved diet.

In one embodiment the grounding device is a wristband with attached metal plate which in use contacts user skin, and wherein an attached wire is in continuity to plate, and thence connected to a plug suitable to be plugged into ground socket and transmit earth energy to user. Thus the grounding device in use transmits earth-ground negative energy to be consumed into body of the person wearing said wristband. Such grounding, as users are herein advised and enabled to undertake, is known to increase zeta potential on circulating red blood cells of such user. This induces lowering of blood viscosity and/or assists to maintain blood viscosity at a healthy low-normal level.

In one embodiment the grounding device is a bedsheet of special design, such as a full size bedsheet or a half-sheet, or the like, and wherein silver or similar conductive metal wires are woven into the linen fabric. These wires, typically in a cross-hatch pattern within the fabric, are connected to an insulated wire via a metal snap. The wire connects to an associated plug. The plug fits into the ground socket of a typical wall socket. Using this bedsheet device, a user by sleeping with some part of user skin touching the metal wire in the fabric, will receive transmitted earth-ground negative energy to be consumed into user's body. Such earth-ground negative energy then proceeds within user's body into user's blood. Regular use as described for user herein, such as every night, can assist in maintaining blood viscosity in a healthful range.

In one embodiment, a user a.) consumes edible bits of composition herein disclosed, such as regularly with daily meals, by b.) first applying said edible bits in amounts and timing as advised onto user's daily food items using a sprinkle-type container/dispenser, and c.) user contemporaneously and regularly uses a blood-thinning device sending earth's negative charge into user's blood to reduce or maintain in healthy range the user's blood viscosity, and d.) user consults expert staff/expert system, such as to calculate gaps in user diet as to consumption of nrf2 activation/inhibitor of acetyltransferase EP300, and e.) user receives related advice via said expert staff/expert system, and f.) expert staff/expert system examines evidence of said user's compliance to applying compositions and consumptions, and g.) said evidence comprises at least reasonable verification user is properly consuming, and h.) said verification further comprises at least one test on user for blood viscosity and/or memory function, and i.) said test results are reported to user, and j.) with permission of user, further reported to at least one of user's relatives.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a chart listing system parts according to an embodiment of the present invention.

FIG. 2 is an illustration of edible bits within a dispenser according to an embodiment of the present invention.

FIG. 3 is an illustration of users using blood thinning devices according to an embodiment of the present invention.

FIG. 4 is a flowchart of biologic actions in body without use of present invention.

FIG. 5 is a flowchart of biologic actions in body with use of present invention.

FIG. 6 is example of informational material associated to the present invention.

FIG. 7 is list of steps of the present invention.

FIG. 8 is example of an algorithm within the present invention

### DETAILED DESCRIPTION OF THE PREFERRED EMBODIMENT

The following description is provided to enable any person skilled in the art to make and use the invention, and sets forth the best modes contemplated by the inventor of carrying out his invention. Various modifications, however, will remain readily apparent to those skilled in the art, since the general principles of the present invention have been defined herein to specifically provide a rapid corrective system for preserving cognitive function during aging, in particular memory capacity—i.e. normal declarative and working memory.

The word “a” is defined to mean “at least one.” The word ‘his’ is defined to mean ‘his/her’. The words ‘chemically pure’ are defined to mean ‘of relatively high degree of purity’ such as 98% pure or the like. ‘Nrf2 activator’ is defined as a substance or formulation capable of acting within a cell to result in increased nrf2 activity, such activity inducing expression of genes within nucleus of said cell, thus inducing a significant and protective rise in the level of natural anti-oxidants within the said cell. Nrf2 activators typically act via the nrf2/KEAP1 pathway and/or nrf2/ARE pathway. Among the known nrf2 activators are: phenolic, proanthocyanidin, catechin and flavonoid compounds of cocoa origin, curcumin, such as from the turmeric plant. carotenoids such as zeaxanthin and lutein, as found for example in egg yolk or green leafy vegetables, quercetin, a plant flavonol from the flavonoid group of polyphenols as found in many fruits, vegetables, leaves, and grains, isoquercitrin in quercetin supplements, and resveratrol, a substance in red wine.

Nrf2 substantially-activating food’ is defined as a foodstuff which when consumed is effective with regard to capacity to activate nrf2 and with a wide margin of safety and few side effects. Among such Nrf2 substantially-activating foods are the following: Dark chocolate content of cocoa flavonoids/polyphenols, quercetin as from eating colorful fruits and berries, sulforaphane as produced, for example, from chewing broccoli sprouts and seeds to combine myrosinase and glucosinolate therein, curcumin as in eating curried food, resveratrol as in drinking red wine.

‘Nrf2 substantial-activators’ are defined as substances which when consumed are effective to activate nrf2 to a biologically significant amount per milligram, such activation inducing production of natural anti-oxidants within a cell, and doing so with a wide margin of safety and few side effects. Such nrf2 activators include extract of cocoa polyphenols, quercetin extract, curcumin as in turmeric, resveratrol, quercetin, isoquercitrin, sulforaphane.

‘Acetyltransferase EP300 substantially-inhibiting food’ is defined as a foodstuff which when consumed is effective with regard to capacity to inhibit acetyltransferase EP300 and with a wide margin of safety and few side effects. Among these are: aged cheese, mushroom, wheat germ, soybean natto, mustard.

‘Acetyltransferase EP300 substantial inhibitor’ is defined as a substance capable, within a living human cell, of substantially facilitating induction of partial autophagy via inhibition of enzyme acetyltransferase EP300 with a wide margin of safety and few side effects. Such action typically disables the binding of acetyl coenzyme A to EIA-associated protein p300. Such disabling leads typically to a beneficial increase in partial autophagy within said cell. Among known acetyltransferase EP300 inhibitors are the natural polyamine spermidine, the cashew shell compound anacardic acid, and salicylate, of which spermidine is generally regarded as safe and effective. The term ‘medicament’ is defined as ‘a healing substance, medicine or remedy’.

‘Polyamines’ are defined as polycationic aliphatic amines, more specifically as natural substances to include spermine and spermidine. Polyamines are known to be found in almost all living organisms, plants and animals. Thus foods typically comprise at least some level of polyamines, though concentration varies widely food to food. In action, polyamines, as positively charged molecules, strongly interact with negatively charged molecules such as DNA, RNA and proteins. In so doing within the human body, polyamines have important consequences for cellular processes involving growth, division, differentiation and survival. Polyamine levels within cells of animals are typically maintained from the diet and from de novo synthesis within the cell itself of polyamine from cellular arginine. The decline of polyamine levels within human cells is associated with age and with various pathologies.

‘Partial Autophagy’ is defined as a cellular function whereby a cell remains alive as its damaged proteins/abnormal proteins within are disintegrated and re-cycled. Full autophagy or apoptosis is when an entire cell undergoes programmed cell death, is removed or disintegrated. ‘Nrf2’ is defined as a protein normally present in cytoplasm of cells which is linked to response to oxidative stress. Typically, during inactive state, nrf2 is bound to a protein known as KEAP1. When an oxidative stress occurs in the cell, KEAP1 protein may be caused to release nrf2. Nrf2 can then proceed into cell nucleus. Within cell nucleus, nrf2 can increase the expression of certain genes, including genes that produce natural anti-oxidants. ‘Neuron damage’ herein is defined as ‘damage to neuron and/or glia’. ‘Brain cell’ is herein defined as ‘neuron and glia’ Polyamines in brain concentrate in glia, and concentrated polyamines are found also in synaptosomes, i.e. isolated synaptic terminal samples from a neuron, such as are mechanically produced for study purposes. Current evidence suggests that polyamines are synthesized by neurons, released into the extracellular space, and taken up by pre-synapse and nearby glial cells (Pharmacological Research Volume 112, October 2016, Pages 99-118). However, this does not mean that polyamines do not interact with or modulate glial cell activity.

‘Aged cheese’ is defined as non-pasteurized cheese cured in a controlled environment for at least six months. The category of spermidine-rich aged cheese typically includes cheddar, brie, parmesan, gruyere and manchego. Research has documented health benefits from aged hard cheese consumption. A 2016 study from the University of Copenhagen reported that regular consumption of such cheese boosted levels of “good” high-density lipoprotein (HDL) cholesterol, believed to protect against cardiovascular and metabolic diseases. Other research has documented the anti-inflammatory benefit of consuming Roquefort cheese. The term ‘supplement/foodstuff’ is defined to mean any nutritional supplement or chemical substance having capacity or feature indicated, and/or any foodstuff comprising at least one substance or capacity linked to feature indicated.

The term ‘expert staff’ is defined to mean human workers as teachers, meal preparers, trainers and other staff, all of whom have demonstrated they are well versed in the herein disclosed method and system. In particular, expert staff are knowledgeable about diet and the formulations herein such that they can adjust consumption choices and advice to the benefit of users. The term ‘expert system’ is defined as a computer and associated software to enable a process whereby an algorithm is applied to solve a problem. The term ‘formulation’ is defined to mean ‘the putting together of components in appropriate relationships to achieve a desired biologic action, according to a formula’. The term ‘gap in user diet’ is defined to mean a deficit or reduction in user consumption from his own diet on average as compared to ideal or population study diet. For example, a gap of nrtf2 activators or gap of inhibitors of acetyltransferase EP300 in user, is/are as compared to average consumption respectively of nrf2 activators and/or inhibitors of acetyltransferase EP300 as consumed on average by a general population reported in peer-reviewed published study.

The term ‘at least reasonably verifiable’ is defined to mean verifying consumptions of user as to compositions herein disclosed, and/or verifying grounding device use by user, and wherein such verifying is accomplished at least by one of the following four general ways: a.) an expert staff member/expert system with capacity then observes and confirms consumption, and/or b.) a user orders and continues to order such consumables such that consumption is reasonably inferred, and/or c.) a user reports, such as to expert staff member/expert system data input acknowledgment of such consumptions by user, and/or d.) a user is tested, such as by expert staff member or by input to expert system, with results of such tests at least tending to confirm likelihood of said consumption by user. Such testing can comprise test of blood viscosity and/or memory testing year by year, or the like.

‘Facilitating’ or ‘Assisting’ is defined herein as generally making it easier, and thus more likely that a user will use/consume the compositions and use the grounding device(s) of the instant invention, such as per advice. Thus, facilitating by expert staff/expert system, or assisting promotes likelihood of user's successful participation long term in the protocol/invention herein disclosed. Long term participation improves user's chance to preserve cognitive function over the aging years. Examples of ‘facilitating’ and ‘assisting’ within instant invention comprise:
a) advising user in use of consumables, such as in selection available among compositions of edible bits, in particular with regard to formulating/choosing/consuming a formulation capable to fill in the gaps in a user's diet as to nrf2 activators and inhibitors of acetyltransferase EP300;
b) providing user with grounding device, and instruction on use;
c) providing user with information about testing, such as for blood viscosity and tests of memory;
d) encouraging user to continue consumable use over time, including to offer user a discount, a promotion prize or the like as user completes use of protocol for 6 consecutive months, and the like;
e) testing user memory and/or blood viscosity;
f) demonstrating to user the proper use of expert system;
g) at least reasonably verifying user consumption has occurred/is occurring;
h) monitoring a user and reporting results, such as to user and/or, with permission, to user group/a relative of user;
i) adjusting/installing/replacing grounding device for user; and,
j) testing ground plug connection to ground for user.

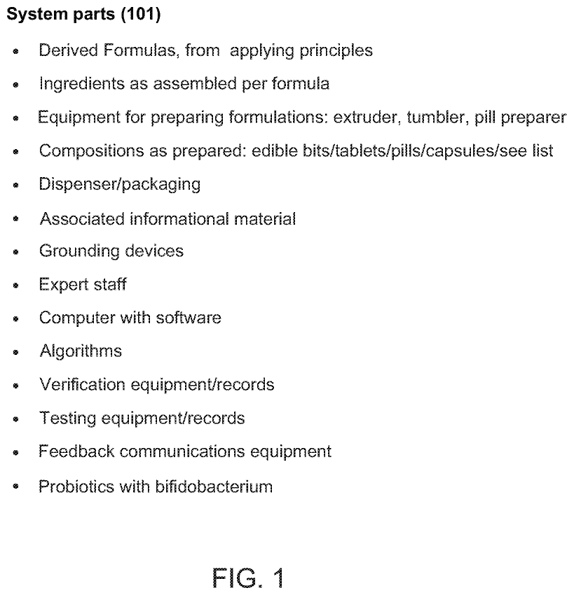

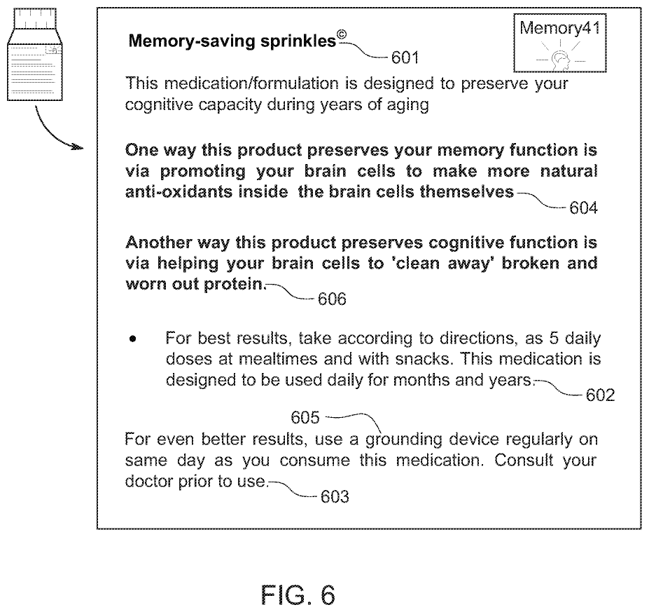

In FIG. 1, system parts 101 comprise formulas for formulation of compositions, wherein said formula comports with principles of preserving cognitive capacity by boosting beneficial natural pathways activities within neurons, ingredients for said formulations assembled to organize composing said compositions, equipment for said composing, compositions thus formed, such as edible bits/tables/pills of various compositions per formulation principles herein disclosed, dispenser/packaging, as with associated informational material (see FIG. 6), at least one grounding device, expert staff/expert system computer/software to assist/facilitate users, computer with purpose-built software as for orders and feedback and other functions herein, algorithms such as for calculating total amount consumed in diet daily/analyzing gaps in user diet regarding nrf2 activator supply/supply of inhibitors of acetyltransferase EP300, verification computer and communication technology/materials and records, testing materials and records, probiotics comprising Bifidobacterium.

In one embodiment, the target level for nrf2 activators is approximately the 67^{th} percentile of population daily consumption of substantial-activators of nrf2, as found in published peer-reviewed population studies. Also in this embodiment simultaneously, the target level for daily consumption of substantial-inhibitors of acetyltransferase EP300 is approximately the 67^{th} percentile of population, as found in published peer-reviewed population studies. In meeting these simultaneous goals for user, the instant invention supplies to user compositions which if consumed by a user who currently is only consuming diet wherein each of these substances is at the average amount of lowest third of said population, will rise to top third in daily consumption respectively. This occurs because said compositions are herein disclosed to be formulated to comprise, in one embodiment, daily supplement content in the range of three to five times standard deviation amounts, per study publication, of both substantial-activators of nrf2 and substantial-inhibitors of acetyltransferase EP300. Said user thus consuming said compositions daily as herein disclosed, is thus likely to be boosted to the 67% percentile of consumption or slightly higher, as compared to the population study, as depends on user starting point and continuing diet.

In one embodiment, the user communicates with an expert staff member and receives assistance pertaining to system components comprising ordering, standing orders, deliveries, use of grounding device, instructions, adjustments, verifying consumption, arranging testing, and the like.

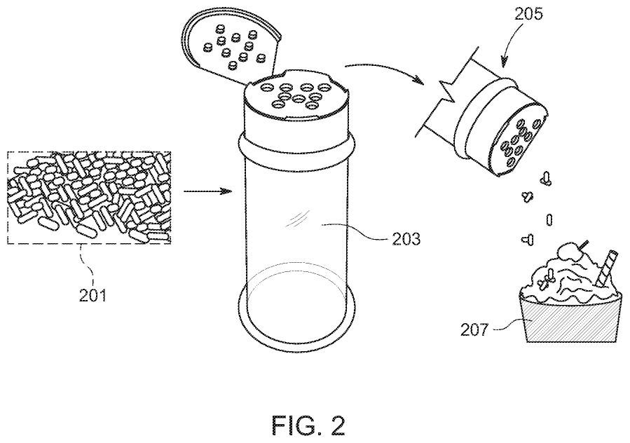

In FIG. 2, a composition of cocoa and wheat germ extract 201 configured as small edible bits is illustrated. In one embodiment, the bits or sprinkles are carried inside a shaker 203 container with a top closed during carrying. When consuming a fast food meal or snack, such as milkshake or pie, user applies a sprinkle of bits from dispenser onto foods 205, shaking out the advised number of bits of said formulation to provide a divided dose as herein disclosed, and 207, then user consumes said divided dose of bits as milkshake and sprinkles together in same mouthfuls. It should be understood, that the construction and shape of the shaker, and of the bits, may vary without departing from the scope of the invention.

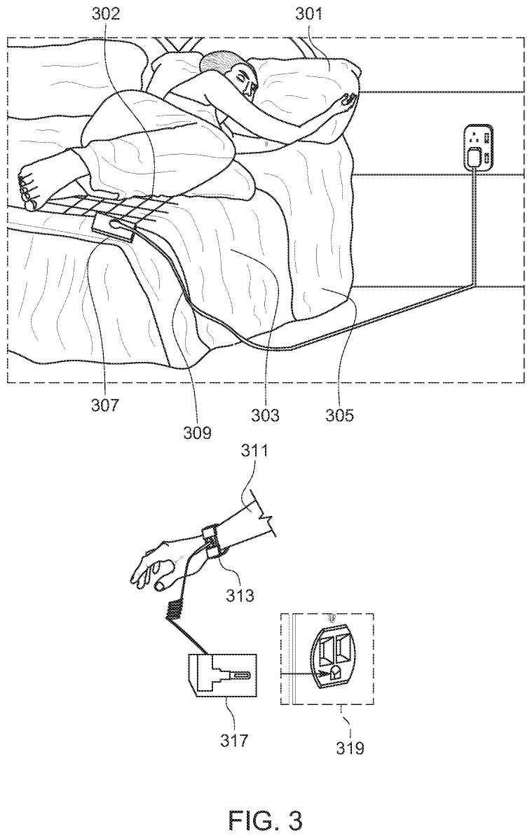

In FIG. 3, examples of two users, each using a grounding or blood thinning device are illustrated. Such devices carry earth-ground negative energy into user's body, and enable thinning of blood. In the top left aspect 301 a woman is in bed 305 sleeping. The bedding 303 has a ‘half-sheet’ at surface of bedding 302 of grounding or blood thinning device type. In one embodiment, the half-bedsheet comprises a fabric with interwoven silver metal threads in a cross weave pattern 302. Such threads of conductive metal serve to convey earth negative charge into body of sleeping user, via contact of skin of woman to thread metal in fabric, which metal threads have contact to metal snap beneath insulation 307 of patch at bottom right of half-sheet. In one embodiment, the metal snap on the underside is in contact to the sheet threads of metal in the fabric, and on the upper aspect of the snap, the metal of snap is in contact to a metal wire 309 within insulation. Said metal wire is in contact to a plug which is plugged into ground plug. Thus, a flow of ground negative charge flows from earth, through wire and snap, into bedsheet metal threads, and hence into the woman's body and blood. This occurs whenever her skin is in contact and the plug is plugged in. This contact to ground enables negative charge from the earth to move into user's blood, and thence onto user red blood cell surface membranes. This increases zeta potential on red blood cells, and thus facilitates blood thinning. With regular use, such a grounding effect can advantageously enable in a user at least a trend towards low-normal blood viscosity.

Still referring to FIG. 3, at the bottom right, a user's hand and lower arm 311 is illustrated. In one embodiment, the user has a wristband 313 on the wrist. In one embodiment, the band has a metal aspect beneath insulation, which metal is in continuity to the underside metal aspect of the snap on the wristband which is in contact to the skin of the user's wrist. The metal within the metal snap is in continuity to the wire metal beneath insulation, which metal of the wire is in continuity to the metal of the plug 317, which the plug is shown adjacent to the ground socket 319 for which it fits. When said wristband plug is properly plugged into ground socket and as the user is properly wearing the wristband, then the ground negative charge flows from earth into woman's blood, and thence onto user's red blood cell surface membranes. This facilitates a trend in user towards low-normal blood viscosity.

Science teaches that a key pathway by which nature allows anti-oxidants to be produced within a cell is via nrf2 activation. Nrf2 is a protein normally present in cytoplasm of cells. Typically, during inactive state, nrf2 is bound to a protein known as KEAP1. When an oxidative stress occurs in the cell, KEAP1 protein may be caused to release nrf2. Nrf2 can then proceed into cell nucleus. Within cell nucleus, nrf2 can increase the expression of certain genes, including genes that produce anti-oxidants. The user of the instant invention is optionally informed by expert staff/expert system about such details, as a means to encourage the user to consume the edible bits of the instant invention per schedule. Consumption supplies user's brain cells with addition activator of nrf2 to fill dietary gaps in user.

Among nrf2 activators, cocoa has special features. Cocoa comprises minerals and flavonoids. Scientific studies have demonstrated benefits to cardiovascular health from cocoa flavonoids, such as polyphenols. Scientific studies have also demonstrated that mineral deficiency often accompanies dementia, such as manganese deficiency frequently found in the serum of Alzheimer's disease patients. Manganese is needed by the cell to make anti-oxidant SOD2. Cocoa flavonoids and minerals thus each have the potential to prevent or slow cognitive decline associated with aging, via filling gaps in cell level of natural anti-oxidants.

A related mechanism by which nature's natural pathways allow brain cells to preserve function is by induction of partial autophagy. Although the word autophagy literally means ‘cell eating itself’, the type of autophagy needed in the brain is partial autophagy. To preserve cognition and in particular to preserve memory, it is necessary to avoid apoptosis of neurons, i.e. avoid death of neurons. Instead, a partial autophagy within neurons is sought because the connection between neurons is how memory is stored. Partial autophagy is the removal of dysfunctional cell parts and abnormal or misfolded protein from within a cell. Such ‘clean up’, while preserving the neuron alive, is what allows preservation of the memory links of that neuron.

By contrast, and for many organs of the body, replacing a cell, such as a liver cell, with another fully capable liver cell, is of little general consequence. A new liver cell is capable to function in ways similar to a removed old liver cell which underwent apoptosis, i.e. complete autophagy. The same is not true for brain neurons, because it is the connections from one neuron to another which allows recall of memory. When a neuron begins to accumulate broken protein or misfolded protein, such can interfere with memory. Yet it is preferable in terms of preserving memory function and capacity, to replace such parts by partial autophagy, rather than to induce apoptotic full cell death of that neuron.

Recent scientific discovery has proven that a key pathway by which nature enables partial autophagy in the brain to proceed is via spermidine, a polyamine. Cells can make polyamines, within the cell, and diet can supply more to the cell. Human gut microbes can also supply spermidine, such as strains from the genera Bifidobacierium, a genus of gram-positive, non-motile, often branched anaerobic bacteria. In a study using a rat model of Alzheimer's disease, Bifidobacterium in probiotics appeared to play a role in improving memory deficit and inhibiting the pathological mechanisms of Alzheimer's disease in the rats (Appl Physiol Nutr Metab. 2018 July; 43(7):718-726), logically associated with spermidine production by the Bifidobacterium. In humans, recent prospective studies have linked extended longevity of approximately 6 years in humans who had been in the highest third of population for spermidine consumption in food. Additionally, research in drosophila revealed a beneficial effect of spermidine on reducing aging-associated increase of abnormal protein, specifically excess presynaptic vesicles in olfactory neurons in a genetically-engineered fly. Such protein accumulation increased in the fly model progressively as it aged. The progressive increase in broken proteins was associated with progressive decrease in memory function in the fly. When researchers started over using similar genetically-engineered flies, but this time with spermidine feeding during all their lives, the abnormal protein was prevented, and the fly memory in such spermidine-fed flies was preserved over aging. In one embodiment of the instant invention, user is provided probiotics comprising Bifidobacterium, such as Bifidobacterium longum.

In one embodiment of the instant invention, users are enabled to maintain their spermidine consumption at healthful levels over aging by supplementing compositions disclosed herein. Spermidine-rich foods comprise wheat germ, mushrooms, green peas, shellfish, chicken liver, potatoes, pears, and aged cheese. Spermidine as 99% pure, is supplied by Sigma-Aldrich. In one embodiment, compositions herein disclosed comprise extracts of spermidine-rich foods, and/or extracts of spermidine-rich plants, and/or aliquots of chemically pure spermidine.

The oldest living human person on record, Jeanne Calment, was reported to have eaten a 100 g size chocolate bar nearly daily—and she lived to a reported age of 122, until her death in 1997. Similar to that longevity connection, other benefits from cocoa are known to science. For example, the Indians of the San Blas islands off the coast of Panama, in Central America, have a traditional cocoa/banana beverage which they consume several times per day. When medical scientists found these Indians had excellent cardiovascular health, they investigated and determined that the cocoa beverage was a key factor. Blood flow improvements from cocoa parallel the half-life of consumed cocoa flavonoids in the body, typically reaching effective levels after 2 hours and significantly declining by 6 hours. This matches the schedule for drinking of their native beverage by the Kuna Indians—typically 4 cups per day, one every 4 hours or so. In one embodiment of the instant invention, a user is enabled to healthful level of nrf2 activator, such as polyphenols of cocoa.

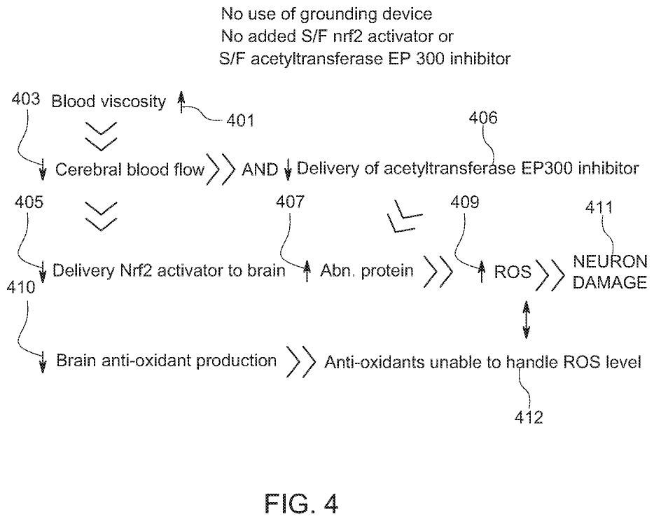

FIG. 4 illustrates the theory of damage to neurons from an unhealthful diet/lifestyle and aging. Typically as such a person ages, blood viscosity rises 401 which causes cerebral blood flow to decline 403. Contemporaneously, the user's poor diet leads to less delivery to the brain of beneficial substances such as cocoa polyphenols or other nrf2 activators 405. Furthermore, in reducing the nrf2 delivery to brain, the poor-diet-choices person avoids foods having nrf2 activators, such as avoiding fruits and berries, as well as avoiding broccoli sprouts.

Poor blood flow to the brain, and poor diet of spermidine-rich foods contemporaneously, together these magnify the decreased delivery to the brain of spermidine 406. Such spermidine inadequate supply, as in a user not consuming green peas or aged cheese, will have inadequate partial autophagy in neurons. Add in the effects of poor blood flow to the brain, and the situation is thus doubly more unlikely to preserve brain cell functions properly. For example, the needed inhibiting of the activity of acetyltransferase EP300 does not happen. Hence, partial autophagy in the cell is prevented, which leads to increased amounts 407 of abnormal, broken and worn out protein to accumulate in the cell. Such abnormal protein is known to boost level of reactive oxygen species 409.

Thus, inadequate anti-oxidants 410 in the brain cells of the unhealthy diet/poor blood flow individual amplifies damage. Such cells face more reactive oxygen species 412, but have fewer anti-oxidants to throw against those damaging species. This leaves unquenched reactive oxygen species floating around in the cell to damage the neuron 411.

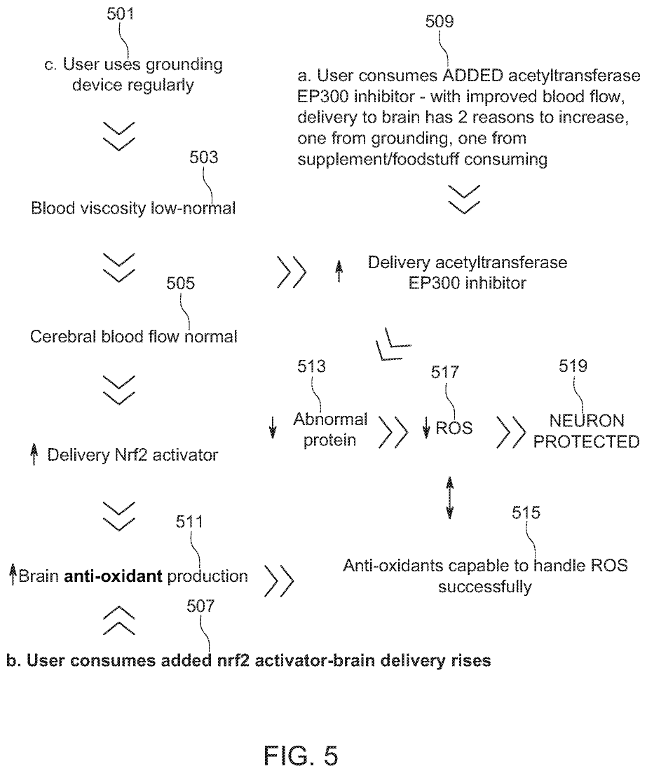

FIG. 5 illustrates the mechanism of protection to neurons from use of the instant invention. Where user receives earth energy via grounding 501, user's blood viscosity trends to the low-normal range 503, which allows cerebral blood flow to increase 505. Meanwhile as the user consumes the edible bits of the instant invention, more nrf2 activators 507 and more inhibitors of acetyltransferase EP300 509 are delivered to blood. With more blood flow to brain and more of said activators and said inhibitors in user's blood, brain anti-oxidant levels rise 511 as simultaneously the abnormal protein is disintegrated via partial autophagy 513. This triple action (improved blood flow to brain, increased nrf2 activation in brain cells to make anti-oxidants, and increased inhibitor which decrease activity of acetyltransferase EP300 in brain cells, hence supplies more partial autophagy) causes circumstance where more anti-oxidants 515 face fewer reactive oxygen species 517 within the brain cell of user. This is the opposite of the dementia-inducing situation faced by brain cells in the brain of the unhealthy diet/high viscosity blood individual. In one embodiment of the instant invention, user is enabled via consumption of the instant invention's compositions, and consumption of earth-ground negative energy via grounding, to preserve cognitive function better for brain cells. This occurs as increased amounts of internal anti-oxidants are able to quench the relatively reduced amounts of internal reactive oxygen species. The neuron is protected 519 from damage. This process as disclosed herein is a day by day, meal by meal process of enabling protection. It is best carried out, as per protocol herein disclosed, as divided doses daily, to match and extinguish the daily aliquot of damaging reactive oxygen species in brain cells. In one embodiment of instant invention, user is enabled to better protect brain cells day by day, and for months and years.

Turning now to the benefits of the instant method and system, it is useful to recall there is a natural circadian cycle in the body, not just for awake and asleep, but also during the day. For example, humans cycle from more alert state to less alert state daily, even during waking hours. For example, people get sleepy after a meal. This cycle of timing is on a different schedule in different organs, such that the brain can rest as the digestive organs gear up.

It seems in today's world, that human brain cells do not have rest periods. Instead, modern humans are stressed and have disturbances in sleep. Such disrupted sleep cycles (Brain, Volume 140, Issue 8, 1 Aug. 2017, pages 2104-2111) and such chronic stress have both been linked by scientific research to increased risk for dementia (Neurobiol of Stress, V8, February 2018, Pages 127-133) as evidenced by increased levels of tau protein in neurons. In one embodiment of the instant invention, combined consumptions as herein disclosed address this stress/disturbed sleep cause of brain cognitive function decline. For example, the advised consumptions herein enable anti-oxidants to be produced within neurons. This is natural and more effective as compared to low level of success with orally supplied pre-formed anti-oxidants. Attempts to use Vitamin E and other oral supplement of anti-oxidants have often yielded poor results. It seems that such a poor result from Vitamin E is because those per-formed anti-oxidants have difficulty arriving in the correct location in cells at the right time, especially if blood flow to brain is reduced due to high blood viscosity. Some experts estimate that a natural anti-oxidant produced inside a cell by gene expression is a thousand times more effective as an anti-oxidant, as compared to a pre-formed anti-oxidant in a pill administered by mouth.

In one embodiment, variations for dose composition, schedule of dosing, amounts for daily dose, and other factors, allows for individualization of protection of cognitive capacity during aging. Furthermore, adjustments in dose, schedule and selection of ingredients can assist a user to stick with the protocol, even as user changes diet. For example, one user might report signs of an allergy while taking a particular nrf2 activator, such as curcumin. Expert staff/expert system advice in such circumstances can assist user to switch to composition with isoquercitrin as nrf2 activator. Where that composition is better tolerated, and thus wherein user is better satisfied, then user is assisted to continue with protocol. This is a benefit to user's cognitive capacity being preserved during aging. It is understood that other substances will be discovered in future to be substantial nrf2 activator and/or substantial inhibitors of acetyltransferase EP300. Use of these later discoveries (Oxidative Medicine and Cellular Longevity, Volume 2016, Article ID 3453926) in formulations as disclosed herein is envisioned. For example, lutein and zeaxanthine are nrf2 activators which are being looked at in new peer-reviewed studies, and appear to have promise (J Int Neuropsychol Soc. 2018; 24(1):77-90).

The instant invention targets a ‘natural anti-oxidants up/abnormal protein down’ approach within neurons to protect cognitive capacity daily in dosed amounts and timing. The contemporaneous use by user of grounding to modulate blood viscosity allows the ‘natural anti-oxidants up/abnormal protein down’ status to be more effectively achieved. In one embodiment, the ‘natural anti-oxidants up/abnormal proteins down’ approach to protecting neurons is applied in a user daily, over years of aging.

In one embodiment, providing information to the public regarding the instant invention approach of ‘natural anti-oxidants up/abnormal proteins down’ is beneficially enabled by specific notice to public. In FIG. 6, for example, a sample of informational material for informing public of benefits is illustrated. Such informational material can be a package insert, a label, a notice online associated to the website offering instant invention compositions, or the like. Such compositions offered, so as to meet needs of public users, are optionally comprised as a choice of edible bits or optionally as choice of tablets, capsules, beverages, condiments, pill, tablet, capsule, food consumable, gelcap, pellet, lozenge, beverage, medicament, feed, chow or the like. In FIG. 6, the name of the product ‘Memory-Saving Sprinkles’ 601, comprises a reference public will likely understand relates to subject of cognition. The reference likely will attract interest of members of the public desiring to preserve their memory. FIG. 6 has a set of instructions regarding schedule of dosing 602, such information also enabling increase in interest of member of the public, as he sees the ease of use. FIG. 6 has a reference to the anti-oxidant benefits to user, and such term is well known to public, hence increasing in public their interest in use of instant invention's consumables 604. FIG. 6 has a reference to grounding device use and advice thereof 603, wherein a member of the public will see the connection, and may have interest to learn more. Mention in informational material of FIG. 6 references as to contemporaneous use 605 of grounding device to days and months and years of consuming by user of comsumables of instant invention, provides member of public with concept of mutual reinforcing benefits. Mention in the informational material of FIG. 6 comprises the term ‘clean-away’ 606, which are words familiar to the public as beneficial. Hence, a member of the public will be more likely to understand how the comsumables enable natural pathways to protect neurons and cognition. This spurs public to use the instant invention. In one embodiment, specific terminology within associated public notice has motivational aspect applicable to increasing likelihood user will better preserve his cognitive capacity during aging years. In one embodiment, the user is provided composition comprising at least one of nrf2 activator and/or inhibitor of acetyltransferase EP300 contemporaneous to providing user said public notice. Optionally, a user is also provided at least one probiotic comprising bifidobacteria.

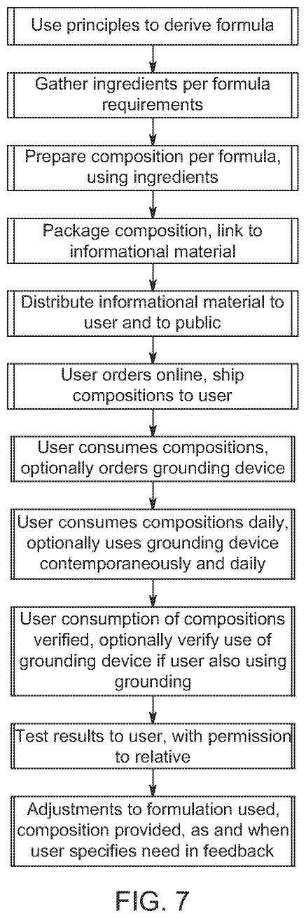

Turning to the edible bits/medication formulations, steps are listed in FIG. 7. For example, in a typical formulation as edible bits, the product is matched to the accompanying shaker or container, in as much as the size of the bit should fit easily through the hole in the shaker, or the size of the bit should easily pour through the open top of a pour-style container. Furthermore, the amounts in a particular formulation, whether medication pill or edible bit or tablet or other, are configured to provide cells in user a relative physiologic balance of gap-filling amounts of significant-activators of nrf2 and significant-inhibitors of acetyltransferase EP300. This balance mimics the balance natural pathways requirements for beneficial mutual activation.

Different from grazing animals who tend to munch gradually during the day, or large ape primates who stroll through jungle munching small bits over hours, modern meals have a different timing. Humans today eat a single large meal at least once a day. This surge of calories to be metabolized is a burden to cells. Such a burden can results in excess reactive oxygen species in cells. In one embodiment of the instant invention, beneficial supply of natural pathway enablers at least seeks to beneficially and timely quench such burden of damaging factors within neurons.

In one embodiment, to beneficially quench such excess reactive oxygen species bursts in neurons, alternate dose schedules and patterns are advised and applied. For example, users might be advised to dose their composition an hour before meals. Optionally, users may be advised to consume formulations of edible bits after the main meal, such as with dessert. Users might be advised to consume more bits to provide a higher dose, such as when the user plans to soon eat, or has just consumed, a high calorie meal.

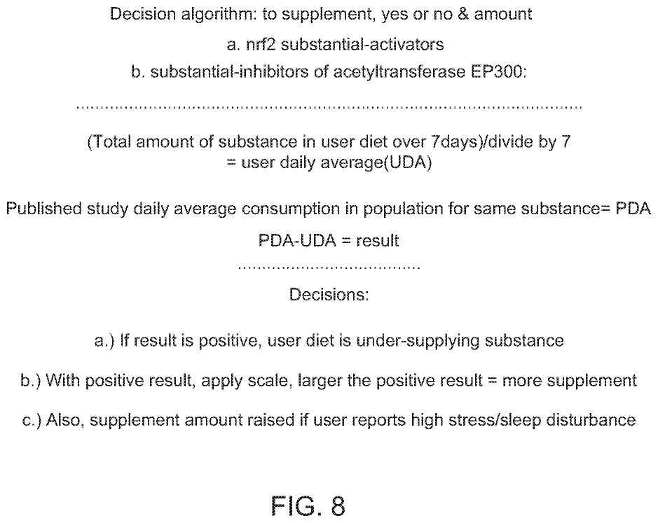

Tailoring composition to gaps in user diet is enabled via use of algorithm as disclosed in FIG. 8. In one embodiment, this algorithm is useful to compute said gaps, and to estimate amount of supplement to apply in composition to account for a gap, for both gaps in nrf2 activator consumption, and gaps in consumption of inhibitors of acetyltransferase EP300. In use, the algorithm operates either manually or is used in conjunction to computer and software, as at least semi-automated. Lists of food are published providing content of various nrf2 activators in each food item, such as by serving. Similarly, lists of food items are published for content of inhibitors of acetyltransferase EP300 in various foods, such as by serving.

Typically, a user provides details of diet over a 7 day period or the like, either providing said data verbally to a member of expert staff, or by user inputting said data into online app of the expert system herein. Based on said data, a computation is carried out per algorithm in FIG. 8. For example, for ‘Total amount of substance in user diet over 7 days’, this is an addition of content of the substance in the servings of the various foods reported as consumed by user during a recent consecutive 7 days. Once this total is computed, the amount is then divided by 7. This yields the user's daily average for the substance in question (UDA).

From published peer-reviewed studies of food items, the daily average consumption for the same substance in a population can be obtained (PDA). Subtracting the UDA figure from the PDA figure produces a result. If the result number is a negative value, this implies the user is receiving in diet at least as much as the average daily in the published study for the population for the substance in question. In that case, a supplement of the said substance can be entertained in the lower range of amount, i.e. not typically a large amount relative to average consumption figure. This comports to principle to maintain level in portion of dose response curve reflecting optimum beneficial effect range. Where the PDA minus UDA result is positive, that implies the user is receiving less in diet of the said substance than the population average. Under such circumstance then, a larger supplement amount is in order. The amount advised will be determined by the size of the positive result value and the dose response curve concept. The amount to supplement is thus determined by judgment, using a sliding scale. This means a results which is a small positive number yields a relatively small supplement amount recommendation of the substance, so as to keep user in optimum range on dose response curve. Where result is a positive but relatively larger number, the amount of the substance advised in daily dose composition for user will be correspondingly larger. For example, a result positive number which is equal to half the average daily consumption amount reported in population study for the substance in question, such circumstance will result in a composition daily dose recommendation for supplementing said substance of approximately half to two-thirds the said substance average daily consumption amount. This places the daily dose at the level of super-centenarian intake or the like. Where the user also reports stress in life at the moment, and/or sleep disturbance, typically the advice is to increase the amount of the substance in advised supplement, at least temporarily. In one embodiment, where high stress level is reported by user, and/or sleep disturbance is reported by user, an increase for recommendations for doses of nrf2 activators and inhibitors of acetyltransferase EP300 may comprise 25% more in moderate stress reports, or up to 80% more, corresponding to higher level of reported stress and/or extreme sleep disturbance.

### EXAMPLES OF FORMULATION

Turning to the formulations, 5 examples of formulations, and resulting compositions, are disclosed herein below. Each example is configured as mid-strength in total daily dose. Although other strengths are possible within the instant invention, the mid-strength dose is adopted for these examples because of the dose response curve nature of supplying drugs and supplements. Simply stated, this concept holds that under-consumption and over-consumption of a particular substance can each carry risks. Furthermore, each of the examples is configured in dose as 5 equally divided doses spread over the day. Other configurations of dose schedule are possible within the instant invention; however the 5 divided doses over a day match reasonably well to when calorie burden enters the user's circulation from meals/snacks. In one embodiment, the timing and dose of nrf2 substantial-activators and the timing and dose of substantial-inhibitors of acetyltransferase EP300 correlate to user intake of calories during the day. Thus, optionally for example, with user having 6 intakes per day of calories, a corresponding schedule of daily doses and amounts in compositions herein is disclosed as: a mid-size dose consumed with breakfast, a small dose consumed with mid-morning snack, a mid-size dose with lunch, a small size dose with afternoon snack, a large size dose with dinner, and a small size dose with evening snack.

In the first two examples below, the active ingredients in edible bits are cocoa polyphenols and a spermidine. The sources from these differ in example #1 as compared to the sources in example #2.

In one embodiment, the procedure for selecting nrf2 activator amount, and/or acetyltransferase EP300 inhibitor amount for formulations comprises 4 steps:
1) Establish daily dose amount required;
2) Establish the molecular form suitable;
3) Establish safety of said dose amount/molecular type for daily use; and,
4) Apply selected molecular form and appropriate dose mathematically within formulation.

Regarding the first part of this procedure, i.e. establishing the daily dose amount, said amount is typically derived using one of following methodologies: Analyze a published peer-reviewed study of general population of humans regarding average daily consumption in that population for a particular nrf2 activator or a particular inhibitor of acetyltransferase EP300. Where the study also provides the standard deviation for the said average daily consumption, that standard deviation figure is used in setting amount in the formulation. For example, the daily amount in formulation is appropriately set at 3 times to 6 times the standard deviation published as to daily intake within study. This method is used in reference to spermidine in examples below.

Some population food consumption studies do not provide the standard deviation figure, however by analyzing a published peer-reviewed study of general population of humans for average daily consumption amount for a particular nrf2 activator or a particular inhibitor of acetyltransferase EP300, the data supplied therein can be used as follows. An appropriate level to set for the average daily consumption amount in instant invention composition daily total is the average daily consumption amount as listed in the study for nrf2 activator and/or inhibitor of acetytransferase EP300. By supplementing using the average amount for daily consumption published, the supplement will enable those users low in consumption to at least reach mid-range using the supplement. This method is used in reference to quercetin in example below.

Where neither standard deviation nor daily average amount from diet in population is available for a particular nrf2 activator and/or inhibitor of acetytransferase EP300, it is useful to analyze published peer-reviewed supplementation studies. In such studies, typically a group of human participants are given a higher dose than might be easily obtained from diet. These are typically short term studies. Where results of the study are seen to indicate benefits to study participants without significant side effect, the study is further analyzed for dosage details. Because such short term studies tend to apply a larger dose than would be appropriate as a daily dose over years, judgment is applied. The dose in the study is typically reduced when setting composition daily total, such as by multiplying by a factor. The factor is selected based on judging safety and effectiveness, based on research. Thus, a study dose is typically adjusted downward by multiplying study daily dose by a factor that varies from ⅔ to 1/50. This method was used for further verifying dose of quercetin/isoquercitrin in example below.

### EXAMPLE 1

The first embodiment example has spermidine from wheat germ extract as inhibitor of acetyltransferase EP300. The first embodiment example has raw organic cocoa as source of polyphenols which serve as nrf2 activator. For the spermidine dose of the first example, the first methodology is used, i.e. peer-reviewed published population study on humans was analyzed (HEALTH 2 (2010) 1327-1334). In that study, researchers reported average daily consumption in food of spermidine for Asian countries and for European countries, including standard deviation. Using an average of standard deviations from this report (3.77+5.45)/2 provides 4.62 micromoles per 1000 calories of food. In respect of the fact that usual diet in Western populations per day is approximately 2000 calories, calculation multiplies standard deviation (4.61 micromoles) times 6 standard deviations to yield the appropriate daily total in composition as supplement, i.e. 27.66 micromoles of spermidine in daily dose total. Thus, for the first example, 28 micromoles spermidine is selected as daily dose amount. Converting 28 micromoles spermidine into micrograms of spermidine requires multiplying by the gram molecular weight, i.e. 145. To calculate the amount in micrograms of spermidine in daily divided dose, as for the first example, it is necessary then to divide by 5. Thus the calculation is (28×145)/5 which yields 4060 micrograms pure spermidine per day, or 5 divided doses, each comprising 812 micrograms spermidine.

Turning now to the cocoa component for the first example, reference is made to a published report regarding low dose habitual cocoa consumption (JAMA 2007 Jul. 4; 298(1):49-60). In this peer-reviewed published report, beneficial results were noted from a randomized, controlled, investigator-blinded, parallel-group trial involving 44 adults aged 56 through 73 years (24 women, 20 men) with untreated upper-range prehypertension or stage 1 hypertension without concomitant risk factors. Study participants received 6.3 grams per day of cocoa, randomly selected to be either polyphenol rich cocoa (study population) or polyphenol poor cocoa (control group).

The study results indicates that such dark chocolate polyphenol-rich cocoa intake mildly reduced mean systolic blood pressure (P<0.001) from baseline to 18 weeks, and mildly reduced mean diastolic blood pressure by (P<0.001) without adverse changes. As a result, hypertension prevalence declined from 86% to 68%. The blood pressure decrease was accompanied by a sustained increase of the natural anti-oxidant in the study group, S-nitrosoglutathione (P<0.001). White chocolate (polyphenol-poor cocoa) intake caused no changes in BP or plasma biomarkers. Thus polyphenol-rich cocoa appears to be a potent nrf2 activator. Starting then with the study dose of 6 grams cocoa per day, formulation herein reduces the study dose by multiplying by factor of ⅔×6=4 grams per day of polyphenol rich cocoa. This amount is selected for total daily dose, hence divided by 5 for divided dose: ⅘=0.8 grams polyphenol-rich cocoa is to be formulated within edible bits as follows (In this example, a spermidine-rich plant extract is used wherein concentration is 812 micrograms spermidine per 1 milligram extract from wheat germ):
⅕×4 grams cocoa=0.8 grams cocoa per each of 5 divided doses,
⅕×4060 micrograms spermidine=812 microgram spermidine per each of 5 divided doses.

As mentioned, the size of bits can be configured to match the shaker/pourer as to size of bit versus container size of hole. In the first example, the edible bit is configured accordingly to fit a shaker which accommodates 5 mgm weight/size bits. In this configuration, the 5 equally divided doses each comprise 801 milligrams of ingredients as:
Divided dose: 800 mgm cocoa and 1 mgm spermidine-rich wheat germ extract, then 801/5 yields the weight of each divided dose as 160.2 mgm. As 5 mgm bits, the number of Formulation #1 bits per each of 5 equal divided doses=160.2/5=32 bits per each of 5 divided doses during day.

Each 5 mgm bit of Formulation #1 would thus comprise approximately:
4.97 mgm cocoa and 0.03 mgm of said spermidine-rich plant extract
Other ingredients such as preservatives, sweeteners and taste enhancers can be added.

### EXAMPLE 2

The second example includes a spermidine source as aged cheddar cheese. The source of the nrf2 activators is a polyphenol extract of cocoa. Per peer-reviewed published study, the amount of spermidine in aged cheddar is reported to be approximately 200 micrograms spermidine in 1.0 gram aged cheddar. To achieve 812 micrograms spermidine in each of the 5 divided daily doses requires 4 grams cheese approximately in each equally divided dose.

Addressing the cocoa extract, a recent publication details pressurized hot water extraction methods to obtain polyphenol-rich extracts from cocoa and cocoa nibs (August 2017, V10, I8, pg 2677-2691). Using these extracts enables dry weight of the extracted polyphenols to average approximately 25 to 30 milligrams polyphenols per gram of starting cocoa/nibs. Thus in the formulations, the amount of polyphenol by weight can replace an equal amount of raw cocoa by a factor of approximately 1/37 gram extract equals same nrf2 activator activity as 1 gram weight of polyphenol-rich cocoa.

Thus, where 800 mgm cocoa was used in the first example for each of 5 divided doses, the requirement of polyphenol extract is 800/37 or 21.6 mgm cocoa per divided dose in the second example. Meanwhile, 812 micrograms spermidine from aged cheddar cheese requires (812/200) grams, or 4.06 grams of cheddar cheese.

Divided dose: 21.6 mgm cocoa and 4.06 grams cheddar cheese. At weight of 100 mgm per bit, the number of bits required in each divided dose is 4082 mgm/100 mgm=41 bits. Each 710 mgm bit of Formulation #2 would comprise approximately:
0.53 mgm cocoa extract and 99.4 mgm of aged cheddar cheese;
Other ingredients such as preservatives, sweeteners and taste enhancers can be added.

### EXAMPLE #3

In the third example, quercetin and cheddar cheese are included. The daily dose of nrf2 activator Quercetin/isoquercitrin is determined. In the establishing of amount, the goal is to boost consumption by approximately the average daily intake as established in a peer-reviewed published population study. In the case of quercetin, the average consumption of quercetin in food has been reported in Japan (Nutrients. 2015 April; 7(4): 2345-2358) to be approximately 16 mgm per day. Therefore a daily dose amount for quercetin at approximately 20 mgm is selected, effectively doubling the estimated average daily amount.

In establishing the molecular form suitable, reference is made to natural sources. In the case of quercetin, the natural form found in leafy vegetables, broccoli, red onions, peppers, apples, grapes, black tea, green tea, red wine, and some fruit juices is isoquercitrin. This is the molecular form used also in most commercial supplements of quercetin.

In establishing the safety and amount for quercetin in daily dose herein, reference is made to studies of quercetin in humans. In the case of quercetin, a recent study (Exp Ther Med 2017 Apr. 13(4): 1353-1359) established that isoquercitrin activates nrf2 via the ERK1/2 pathway. One of the first clinical studies with quercetin was a double-blind study in nonbacterial chronic prostatitis (Urology 1999; 54(6):960-63) wherein patients received 1 gram per day (500 mg twice daily). That dose was well tolerated, and more than 67 percent of patients had an improvement of symptoms of at least 25 percent. Meanwhile, the most popular and widespread application for quercetin is in beverages designed for athletic performance. In a number of clinical trials, quercetin has been shown to improve mental/physical performance and reduce infection risk. However, the instant invention is not a short term trial design, but rather a dosing designed for daily application over years to preserve cognitive function. Hence, rather than 1 gram, a preferred dose of quercetin for instant invention daily dose is judged to be appropriate at amount approximately equal to 1/50 the prostatitis study dose, which approximates the average consumption as found in said general population study, of 16 mgm. Thus 20 mgm isoquercitrin is chosen as daily total dose of nrf2 activator . . . \

The nrf2 activation is supplied by isoquercitrin, and the inhibitor of acetyltransferase EP300 is cheddar cheese spermidine, the formulation is calculated in 5 divided doses as follows:
As in Formulation #2, each of 5 divided doses during the day each require 812 micrograms spermidine from aged cheddar cheese. This equals (812/200) gram of cheddar cheese per divided dose. For isoquercitrin, 20 mgm isoquercitrin is selected for daily dose (approximately doubles average consumption per day in general population). Thus for each of the 5 divided doses, 4.0 mgm isoquercitrin is required.

Divided dose: 4.0 mgm isoquercitrin and 4.06 grams cheddar cheese. At weight of 100 mgm per bit, the number of bits required in each divided dose is 4064 mgm/100 mgm=41 bits Each 100 mgm bit of Formulation #3 would comprise approximately:
0.098 mgm isoquercitrin and 99.9 mgm of aged cheddar cheese.

### EXAMPLE #4

The wheat germ extract has 812 micrograms spermidine per 1 mgm extract. Applying for spermidine, the calculation in Formulation #1 above, and applying for the isoquercitrin, calculation in Formulation #3 above yields for Formulation #4 as follows:
20 mgm isoquercitrin for daily dose, i.e. the Nrf2 activator, and,
4060 micrograms spermidine for daily dose, i.e. 5 mgm of same extract as in Formulation #1,

Comprising this daily dose then as 5 divided doses, and comprising each divided dose as a tablet, thus the 50 milligram tablet comprises 90% filler/preservative, 1 mgm spermidine-rich extract plus 4 mgm isoquercitrin.

### EXAMPLE #5

In the fifth example, spermidine from chemically pure source serves here as sole active component. Some users might enjoy consuming berries and fruit (quercetin) and also dark chocolate (polyphenols), but might not like the taste of spermidine-rich foods such as broccoli, aged cheese, wheat germ or natto. In one embodiment, when such users are identified by diet analysis, expert staff/expert system recommends composition comprising inhibitor of acetyltransferase EP300 as sole active ingredient. To accomplish the formulation thereof, a pure source of spermidine is used.

A daily total dose of spermidine at least approximating the range of 4060 micrograms spermidine is formulated and provided to user, optionally in 5 equal doses, optionally advised to user to consume with meals and snacks during day. Higher doses can optionally be used, such as with user experiencing disrupted sleep or experiencing stressful times. Such spermidine daily dose is typically configured as pill, produced according to parameters well known in the art. The spermidine pill, in 5 evenly divided doses, is 4060/5=812 micrograms of pure spermidine in pill form, with appropriate preservatives and fillers. Such a strength pill is advised as, for example one with breakfast, one-half pill with mid-morning snack, one pill with lunch, one-half pill with mid-afternoon snack, and two pills at dinner. For such user, optional use of grounding is typically also advised.

Turning now to verification, the consumptions by users herein are reasonably verifiable as discussed above. Such verification is optionally automated wherein a software program analyzes ordering pattern of a user to determine satisfactory supply of compositions which match filling the gaps which have been determined to exist in user's diet for nrf2 activators and inhibitors of acetyltransferase EP300.

In one embodiment, if user diet inadequate in spermidine also then changes to comprise a diet inadequate in spermidine and also inadequate in consumption of nrf2 activators, i.e. inadequate in consumption of fresh fruit, berries, polyphenol-rich cocoa and chocolate, then the composition advised is adjusted accordingly. In this manner, user dietary changes continue to be accommodated herein over the long term. Thus user continues to receive and consume compositions useful to boost user's protection of cognitive capacity through years of aging. Additional formulations are possible herein, such as for newly published data on substances suitable to fit gaps found in diet of users. Such additional formulations typically comprise substantial-activator substances for nrf2, and substantial-inhibitor substances as to acetyltransferase EP300.

Among the current choices of substantial nrf2 activators are dark chocolate, curcumin, quercetin, sulforaphane such as that generated by combination of myrosinase and glucosinolate as from broccoli sprout/seed powder, lutein and zeaxanthin. Among the current choices of foods/substances having substantial activity to inhibit acetyltransferase EP300 are aged cheese or its spermidine-rich extract, natto or spermidine-rich extract of natto, wheat germ or spermidine-rich extract of wheat germ, mushroom or spermidine-rich extract of mushroom, mustard or spermidine-rich extract of mustard, and pure spermidine as for example from chemical/pharmaceutical manufacturers.

The instant invention optionally applies small balanced doses daily of said inhibitors as sole active ingredient. This approach is based on studies published and known aspects of physiology. One useful study in this regard was research on spermidine use in mice exposed to bleomycin. Bleomycin, a toxin used to treat cancer is known to cause the side effect of fibrosis of the lung. The study demonstrated that pre-treatment in mice with spermidine could reduce or prevent the damaging protein from accumulating in the mouse lung. However, once the scar tissue was established in the lung, subsequent dosing with spermidine, even at higher dose, did not remove it. This finding suggests that use of the instant invention as daily balanced physiologic doses is appropriate and natural protective pathway reinforcing.

In one embodiment, the instant invention is applied to non-human mammals, wherein the dosage and timing is adjusted to match needs in said size animal. In one embodiment, edibles containing nrf2 activators and inhibitors of acetyltransferase EP300 are adjusted to body weight needs of animal. The recent discovery of the abnormal proteins of Alzheimer's disease in brains of some dolphin and chimps and other animals (Alzheimer's and Dementia, V14, I2, pg 195-204) is noteworthy. These facts support the logic of applying the instant invention to animal food, medication for animals, supplements for animals and the like. These compositions are contemplated, and are within the instant invention.

Ideally, the edible bits herein configured as sprinkles have a thin exterior coating, such as confectioner's glaze and carnauba wax. This factor serves to preserve the contents, extend the shelf-life and prevent bits from sticking together. In other embodiments, the formulations herein disclosed are optionally configured as animal chow, fish food, cat food, bird food, gummies, candies, chewing gums, cheese crumbles, snacks, supplement powders, beverages, ice cream, milkshake, smoothie mix, beverages, pizza topping, cake topping, muffin topping, cookie topping, pie topping, pudding topping, and ice cream topping.

In one embodiment, the procedure for manufacturing said edible bits comprises the steps below:
1) Ingredients mixed, including active ingredients, plus food coloring as selected, shortening as needed, liquid and powder food colorants as needed.
2) Exact quantity of mix then added to water-filled mixing tank; then water in tank is heated as mixture agitated to dissolve mixture.
3) Worker separately uses a dry mixer to which he adds exact quantity of sugar, starch or filler.
4) Add premeasured food coloring as optionally needed, mix thoroughly
5) Water/shortening/ingredients from step 2 added to said dry mixer of step 4,
6) Close lid and begin dough mixing process
7) 15 mins of mixing, open lid to inspect dough, remove if satisfactory
8) Place dough onto conveyer belt heading to extruder machine
9) Dough fed into extruder where the dough is forced through small holes of size fitting desired diameter of bits, dough exits below extruder plate formed as long strands, falling onto moving conveyor belt
10) Humidity in room kept at 50% and temp kept constant, as conveyor belt conducts said strands of dough towards drying tunnel which uses ambient air
11) Ambient air dries dough in tunnel via conveyer
12) Approximately 10 sees after entering tunnel, the stings of dough exit conveyor and fall into a pail
13) Worker sifts strings in pail and inspects to be sure strings are properly dried
14) If dough strings satisfactorily dry, then worker takes pail to tumbler, places dry strands inside, sets tumbler to rotate
15) Tumbler breaks strands into bits, usually takes 10 mins, not more or bits broken into too small bits, tumbler turned off.
16) Sprinkles removed and nozzle sprays the dried small bits with a mix of confectioners glaze and carnauba wax
17) Finished sprinkles are poured onto tumbler table for separation by size. Upper plate shakes and bits of proper or smaller size go through holes in filter, then fall to lower second filter.
18) Second filter then tumbles bits and second filter removes bits which are too small, leaving correct size bits on conveyor belt
19) Sprinkles ride conveyor belt to end, and there fall into a plastic lined packaging tub
20) Worker does Quality control inspection of sprinkles in packaging tub
21) If sprinkles pass inspection they are taken to bottling machine, where the sprinkles are conveyed towards a moving line of bottles
22) Sprinkles pour automatically into bottles, air spray keeps cap area clear
23) Bottles are filled, then machine spins sprinkle-filled bottle and attaches cap
24) Adhesive label is attached to sprinkle-filled bottle by machine

Although the invention has been described in considerable detail in language specific to structural features, and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

What is claimed is:

1. A method for improving brain cell health in a user during aging, comprising steps:
a. providing user at least one dose of a consumable comprising a spermidine.

2. The method of claim 1, wherein said at least one dose of a consumable further comprises at least one nrf2 substantial-activator.

3. The method of claim 1, further comprising a step of providing said user a grounding device to consume earth-ground negative energy.

4. The method of claim 1, further comprising a step of advising said user to consume a Bifidobacterium-comprising probiotic.

5. The method of claim 1, further comprising a step of providing at least one associated notice accessible by public and at least alluding to at least one benefit, and at least alluding to the brain.

6. The method of claim 1, further comprising a step of advising said user to consume daily, for at least 1 week of daily consuming, at least one dose of said consumable.

7. The method of claim 1, further comprising a step of advising said user to adjust dose of said spermidine consumed at one time by reference to amount of calories consumed by user at that time.

8. The method of claim 1, wherein said at least one dose of said consumable is configured as at least one: tablet, pill, capsule, pharmaceutical, confection, sprinkle, edible bit, food, snack, animal chow, feed, additive to food, spread, beverage, condiment, food consumable, gelcap, pellet, lozenge, animal food.

9. A method for preserving cognitive capacity in a user over years of user aging, comprising steps of:
a. calculating gap in dietary intake of said user regarding at least one of: nrf2 substantial-activator, substantial-inhibitor of acetyltransferase EP300, and,
b. providing said user at least one composition at least logically suitable to at least assist filling said calculated dietary gap.

10. The method of claim 9, wherein said at least one composition comprises a spermidine.

11. The method of claim 10, wherein said at least one composition further comprises at least one of list: flavonoid, polyphenol, sulforaphane, quercetin, nrf2 substantial-activator.

12. The method of claim 10, further comprising a step of advising said user to do grounding contemporaneously via a grounding device.

13. The method of claim 10, further comprising a step of advising said user to consume Bifidobacterium-comprising probiotic contemporaneously.

14. The method of claim 10, further comprising a step of providing at least one associated notice, accessible by public, such notice at least alluding to cognitive benefit potential.

15. A method for preserving cognitive capacity of a user during years of aging, comprising steps:
a. providing said user a medicament comprising a spermidine.

16. The method of claim 15, wherein said medicament further comprises at least one of: flavonoid, polyphenol, sulforaphane, quercetin, isoquercitrin, nrf2 activator-comprising plant extract, nrf2 activator-comprising food extract, nrf2 substantial-activator.

17. The method of claim 15, wherein said medicament's spermidine is provided for consuming by said user in divided doses within a day.

18. The method of claim 17, further comprising a step of advising said user, at least in effect, to consume a higher amount of spermidine when user consumes a higher amount of calories.

19. The method of claim 17, further comprising a step of advising said user, at least in effect, to consume a smaller amount of spermidine when said user consumes a smaller amount of calories.

20. The method of claim 15, further comprising a step of advising said user, at least in effect, to time said user's consumption of said spermidine-comprising medicament with regard to at least one of: time when said user experiences stress or time when said user experiences a sleep disturbance.
